SEQUENCE LISTING (5 sequences)

SEQ ID NO: 1 - protein, 140 aa, Nitrosomonas europaea
HGERSQEPFLRMRTVQWYDIKWGPEVTKVNENAKITGKFHLAEDWPRAAAQPDFSFFNVGSPSPVFVRLSTKINGHPWFISGPLQIGRDYEFEVNLRARIPGRHHMHAMLNVKDAGPIAGPGAWMNITGSWDDFTNPLKL

SEQ ID NO: 2 - protein, 151 aa, Nitrosomonas europaea
QAGQSKVAALPVAPNPVSIVITDANYDVPGRALRVTMEVTNNGDIPVTFGEFTTAGIRFINSTGRKYLDPQYPRELIAVGLNFDDESAIQPGQTKELKMEAKDALWEIQRLMALLGDPESRFGGLLMSWDAEGNRHINSIAGPVIPVFTKL

SEQ ID NO: 3 - protein, 140 aa, Nocardioides sp.
HGEESQQAFQRTSTVVFYDVKFSDDTVDVGESVTITGMVRVMKSWPDHTLEPPEMGYLTVSTPGPVFYVQEREMSGEFTPQSVRIEKGATYPFKLVIKARQPGTWHVHPGFGVEGAGTLVGAGKDITVNDTGVFENTVTL

SEQ ID NO: 4 - protein, 147 aa, Nocardioides sp.
QVVRTTPVPLAEEEVSGAVAPEIESIRFNAEADTLTMKLRVENTGAAAVRLQRVQFGDVEFVSPSFASAADPDAQAMTVTPDQAIEPGGSATFTVEIQSEDLIVRSLVPVNEAELRVTGLLFFEDETGEQVVSEVNELTSAILQDFH

SEQ ID NO: 5 - protein, 250 aa, Methylococcus capsulatus
CRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

(19) United States
(12) Patent Application Publication
(10) Pub. No.: US 2024/0209327 A1
(43) Pub. Date: Jun. 27, 2024

(54) NOVEL PROTEIN HAVING METHANE OR BUTANE OXIDATION ACTIVITY

(71) Applicant: KOREA UNIVERSITY RESEARCH AND BUSINESS FOUNDATION, Seoul (KR)

(72) Inventors: Jee Won LEE, Seoul (KR); Yeon Hwa YU, Seoul (KR); Yoo Bin CHOI, Paju-si (KR)

(73) Assignee: KOREA UNIVERSITY RESEARCH AND BUSINESS FOUNDATION, Seoul (KR)

(21) Appl. No.: 18/288,728

(22) PCT Filed: Apr. 15, 2022
(86) PCT No.: PCT/KR2022/005481

§ 371 Date: Oct. 27, 2023

(30) Foreign Application Priority Data
Apr. 30, 2021 (KR) 10-2021-0056794

(51) Int. Cl. C12N 9/02; C07K 14/47; C12P 7/04; C12P 7/16

(52) U.S. Cl. CPC C12N 9/0073; C07K 14/47; C12N 9/0083; C12P 7/04; C12P 7/16; C12Y 114/99039

(57) ABSTRACT

The present invention relates to: a protein having activity for producing methanol or butanol by oxidizing methane or butanol; a microorganism expressing same; a composition for producing methanol or butanol using same; and a method for producing methanol or butanol, wherein the protein is a self-assembled protein comprising a ferritin monomer fused with an ammonia oxidase active domain having methane oxidation activity, or a butane oxidase active domain having butane oxidation activity.

### TECHNICAL FIELD

The present invention relates to a novel protein having methane or butane oxidation activity.

### BACKGROUND ART

Methane monooxygenase (MMO) derived from methanotrophs is a very useful biocatalyst that can catalyze the oxidation reaction of various hydrocarbons (C1-C8) including methane gas under mild conditions at room temperature and atmospheric pressure to produce high value-added products, therefore, global attention is focused on the development of a bioprocess using the above biocatalyst.

Further, as a similar enzyme, an ammonia oxidase (ammonia monoxygenase, AMO) derived from Nitrosomonas europaea, butane oxidase (butane monooxygease, BMO) derived from Nocardioides sp. Strain CF8, etc. are also useful biocatlysts that have a similar mechanism to that of methane oxidase, and can catalyze oxidation reactions for a wide range of hydrocarbons (AMO: C1-C10 chain/halogenated hydrocarbons, mono/polycyclic aromatic hydrocarbons, BMO: C2-C10 chain/halogenated hydrocarbons, some aromatic hydrocarbons). However, basic research on 3D structure, identification of active domains, reaction mechanism, substrate specificity, or the like is still very insufficient.

Currently, methanol and butanol production by a chemical process of methane gas and butane gas is complicated in steps, and involves many problems in technical, environmental and economic aspects such as environmental pollution caused by the by-products (carbon dioxide, syngas, etc.), low reaction conversion rate, high energy consumption due to high temperature and high pressure reaction conditions or the like. Further, for methane gas, methanol production using a small-scale bio plant that can be easily connected to a local gas field is very advantageous in technical and economic aspects, due to problems such as economic degradation due to expensive transportation and storage costs, and occurrence of serious greenhouse effects when leaked.

As a result of efforts to develop bio-processes, metabolic engineering strain improvement of methane-oxidizing bacteria and various hydrocarbon degrading bacteria for the production of high value-added products other than methanol is being attempted, however, there are limitations in the use of genetic engineering tools and difficulties due to unculturable properties of the strain. Further, heterologous expression using industrial strains for mass production of methane oxidase entails a difficulty in expression of water-soluble protein and requires precise interaction of enzyme complexes, whereby no success case of industrial application is reported due to high technical difficulty.

### SUMMARY OF INVENTION

### Problems to be Solved by Invention

An object of the present invention is to provide a protein having excellent methane or butane oxidation performance.

Another object of the present invention is to provide a microorganism expressing the protein.

In addition, another object of the present invention is to provide a composition for preparing methanol or butanol, including the protein or microorganism.

Further, another object of the present invention is to provide a method for production of methanol or butanol using the protein or microorganism.

### Means for Solving Problems

1. A protein including self-assembled ferritin monomers, in which an ammonia oxidase active domain having methane oxidation activity, or a butane oxidase active domain having butane oxidation activity is fused.

2. The protein according to the above 1, wherein the ammonia oxidase active domain is selected from amoB1 (Ammonia monooxygenase beta subunit_domain 1) and amoB2 (Ammonia monooxygenase beta subunit_domain 2).

3. The protein according to the above 2, wherein the amoB1 consists of an amino acid sequence of SEQ ID NO: 1, and the amoB2 consists of an amino acid sequence of SEQ ID NO: 2.

4. The protein according to the above 2, wherein ferritin monomers, in which amoB1 and amoB2 are fused, are self-assembled.

5. The protein according to the above 2, wherein a ferritin monomer fused with amoB1 and a ferritin monomer fused with amoB2 are self-assembled.

6. The protein according to the above 1, wherein the butane oxidase active domain is selected from bmoB1 (Particulate Butane monooxygenase subunit B_domain 1) and bmoB2 (Particulate Butane monooxygenase subunit B_domain 2).

7. The protein according to the above 6, wherein the bmoB1 consists of an amino acid sequence of SEQ ID NO: 3, and the bmoB2 consists of an amino acid sequence of SEQ ID NO: 4.

8. The protein according to the above 6, wherein ferritin monomers, in which bmoB1 and bmoB2 are fused, are self-assembled.

9. The protein according to the above 6, wherein a ferritin monomer fused with bmoB1 and a ferritin monomer fused with bmoB2 are self-assembled.

10. The protein according to the above 1, wherein the ferritin monomer is a human ferritin heavy chain monomer.

11. The protein according to the above 10, wherein each domain is fused to any one selected from the group consisting of: inside α-helix of the ferritin monomer; between adjacent α-helices; N-terminus; C-terminus; A-B loop; B-C loop; C-D loop; D-E loop; between N-terminus and A helix; and between E helix and C-terminus.

12. A microorganism for expressing the protein according to any one of the above 1 to 11.

13. The microorganism according to the above 12, wherein the microorganism is introduced with a vector which includes: a gene encoding a ferritin monomer; and a gene encoding an ammonia oxidase active domain selected from amoB1 (Ammonia monooxygenase beta subunit_domain 1) and amoB2 (Ammonia monooxygenase beta subunit_domain 2), or a butane oxidase active domain selected from bmoB1 (Particulate Butane monooxygenase subunit B_domain 1) and bmoB2 (Particulate Butane monooxygenase subunit B_domain 2).

14. The microorganism according to the above 12, wherein the microorganism is E. coli.

15. A composition for preparing methanol, including the protein according to any one of the above 1 to 11, wherein the protein is fused with an ammonia oxidase active domain.

16. The composition according to the above 15, further including a reducing agent.

17. The composition according to the above 15, wherein the reducing agent is duroquinol.

18. A method for production of methanol, including reacting the composition according to the above 15 with methane gas.

19. A composition for preparing butanol, including the protein according to any one of the above 1 to 11, wherein the protein is fused with a butaneoxidase active domain.

20. The composition according to the above 19, further including a reducing agent.

21. The composition according to the above 19, wherein the reducing agent is duroquinol.

22. A method for production of butanol, including reacting the composition according to the above 19 with butane gas.

### Advantageous Effects

The protein of the present invention has methane or butane oxidation activity.

The protein of the present invention includes a large number of domains having methane or butane oxidation activity, thereby exhibiting high activity.

The composition and method of the present invention can produce methanol or butanol in high yield.

### BRIEF DESCRIPTION OF DRAWINGS

FIG. 1 is a schematic diagram of each vector used in the examples.

FIG. 2 shows results of analysis of the expression rate and cytoplasmic solubility of recombinant proteins including cAMO, AMO-, and BMO-mimics prepared in the examples.

FIG. 3 shows results of confirming that the recombinant protein including cAMO, AMO- and BMO-mimics prepared in the examples forms a self-assembly.

FIGS. 4a-c show results of X-ray absorption near-edge structure (XANES), extended X-ray absorption fine structure (EXAFS), and electron paramagnetic resonance (EPR) spectra analysis of cAMO recombinant protein.

FIG. 5 shows results of confirming the methane and butane gas oxidation activity of the recombinant protein including cAMO, AMO-, BMO-mimics prepared in the example.

FIG. 6 shows results of confirming the 13C-methane gas oxidation activity of cAMO prepared in the example.

### MODE FOR CARRYING OUT INVENTION

Hereinafter, the present invention will be described in detail.

The present invention relates to a protein comprising self-assembled ferritin monomers, in which an ammonia oxidase active domain having methane oxidation activity or a butane oxidase active domain having butane oxidation activity is fused.

The ammonia oxidase active domain may be used without limitation thereof as long as it has an activity to oxidize methane, for example, amoB1 (Ammonia monooxygenase beta subunit_domain 1) and amoB2 (Ammonia monooxygenase beta subunit_domain 2) may be used. Specifically, the amoB1 used herein may include an amino acid sequence of SEQ ID NO: 1, while the amoB2 used herein may include an amino acid sequence of SEQ ID NO: 2.

The butane oxidase active domain may be used without limitation thereof as long as it has butane oxidation activity, for example, bmoB1 (Particulate Butane monooxygenase subunit B_domain 1) and bmoB2 (Particulate Butane monooxygenase subunit B_domain 2), etc. may be used. Specifically, the bmoB1 used herein may include an amino acid sequence of SEQ ID NO: 3, while the bmoB2 used herein may include an amino acid sequence of SEQ ID NO: 4.

In the protein of the present invention, individual domains may be all fused to one ferritin monomer, each domain may be fused to each ferritin monomer, or these domains may be mixed together.

That is, in the protein of the present invention, two ammonia oxidase active domains or butane oxidase active domains may be fused in one ferritin monomer, or one domain may be fused to each ferritin monomer.

The protein of the present invention may further include an electron transfer domain including a flavin adenine dinucleotide (FAD)-binding domain, which is fused thereto.

Methane may be oxidized to form methanol according to a reaction of Equation 1 below. The protein of the present invention may include self-assembled ferritin monomers, in which a methane oxidation active domain; and an electron transfer domain including a flavin adenine dinucleotide (FAD)-binding domain are fused, wherein a methane oxidation reaction may be performed using a reducing agent NADH. In particular, when the reaction is performed in vivo, NADH in the body may be utilized. Accordingly, the use of a separate reducing agent is unnecessary.

CH₄+O₂+NAD(P)H+H⁺→CH₃OH+NAD(P)⁺+H₂O [Equation 1]

The electron transfer domain may include a flavin adenine dinucleotide (FAD)-binding domain.

The FAD-binding domain may be derived from soluble MMO (Methane monooxygenase) (sMMO), and specifically may be a FAD-binding domain of MMOR, which is one of its components.

The electron transfer domain includes a FAD-binding domain, which may consist of only the FAD-binding domain, may further include an additional moiety in addition to the FAD-binding domain in MMOR, may further include at least a portion of 2Fe-2S domain in addition to the FAD-binding domain, and may include a FAD-binding domain and a 2Fe-2S domain. For example, the electron transfer domain used herein may include an amino acid sequence of SEQ ID NO: 5.

As the ferritin monomer in the protein of the present invention, ferritin derived from various organisms may be used, and in the case of vertebrates, a heavy chain or light chain monomer may be used. For example, a human ferritin heavy chain can be used.

The binding site of each domain is not limited as long as it can function in the self-assembled ferritin monomers of the protein, for example, the domain may be fused in any one site selected from the group consisting of: inside α-helix; between adjacent α-helices; N-terminus; C-terminus; A-B loop; B-C loop; C-D loop; D-E loop, between N-terminus and A helix; and between E helix and C-terminus, and, in an aspect of facilitating the function by being expressed externally from the protein, it is preferably fused at C-terminus.

In the protein of the present invention, a linker may be further included between the ferritin monomer and each domain.

As the linker, those known in the art may be used without limitation thereof, for example, S1 (G3SG3TG3SG3), S2 (GKLGGG), and the like may be used.

The protein of the present invention may be, for example, obtained from an organism by transforming a vector which includes: a gene encoding a ferritin monomer; and a gene encoding an ammonia oxidase active domain selected from amoB1 (Ammonia monooxygenase beta subunit_domain 1) and amoB2 (Ammonia monooxygenase beta subunit_domain 2), or a butane oxidase active domain selected from bmoB1 (Particulate Butane monooxygenase subunit B_domain 1) and bmoB2 (Particulate Butane monooxygenase subunit B_domain 2), in the organism, but it is not limited thereto.

The protein of the present invention has a high expression rate in a soluble form in a microorganism, and thus has a high production yield during biosynthesis.

Further, the present invention relates to a microorganism expressing the protein.

The microorganism of the present invention may be introduced with a vector which includes: a gene encoding a ferritin monomer; and a gene encoding an ammonia oxidase active domain selected from amoB1 (Ammonia monooxygenase beta subunit_domain 1) and amoB2 (Ammonia monooxygenase beta subunit_domain 2), or a butane oxidase active domain selected from bmoB1 (Particulate Butane monooxygenase subunit B_domain 1) and bmoB2 (Particulate Butane monooxygenase subunit B_domain 2), so as to express the protein.

In the protein of the present invention, individual domain may be all fused to one ferritin monomer, each domain may be fused to each ferritin monomer, or these domains may be mixed together. As a result, the gene encoding the ammonia oxidase active domain or butane oxidase active domain may be included in one vector or included in two vectors, respectively.

As the vector, an expression vector known in the art may be used, for example, BLUESCRIPT vector (Stratagene), T7 expression vector (Invitrogen), pET vector (Novagen), etc. may be included, but it is not limited thereto.

The vector may further include additional elements known in the art, such as a promoter for protein expression, a tag for isolation/purification, and a transform marker.

The type of microorganism is not limited as long as the vector can be introduced to express the protein, and for example, E. coli may be used.

Since the microorganism of the present invention expresses the protein, it can be utilized to produce methanol by oxidation of methane. When the microorganism of the present invention is used, it is not necessary to add a separate reducing agent for methanol production.

Further, the present invention relates to a composition for preparing methanol or a composition for preparing butanol, which includes the above-described protein or the above-described microorganism.

A protein including self-assembled ferritin monomers, in which the above-described ammonia oxidase active domain having methane oxidation activity is fused, may have methane oxidation activity. Similarly, a protein including self-assembled ferritin monomers, in which the butane oxidase active domain having butane oxidation activity is fused, may have butane oxidation activity. Since the microorganisms described above express the protein, the composition of the present invention may include the above protein and oxidize methane or butane to produce methanol or butanol.

The production of methanol or butanol may be performed by treating the composition with methane gas or butane gas.

The composition of the present invention may further include a reducing agent used for methane or butane oxidation. The reducing agent may be, for example, duroquinol.

Further, the present invention relates to a method for production of methanol or butanol which includes reacting the above-described composition with methane gas or butane gas.

Methanol or butanol can be produced by reacting the composition according to the present invention with methane gas or butane gas to oxidize methane or butane, and this process may be conducted by injecting methane gas or butane gas into the above-described composition and performing an enzymatic reaction.

Conditions for methanol or butanol production are not particularly limited and, for example, may be performed under conditions such as temperature and pH at which the above-described protein or microorganism exhibits appropriate activity.

### Example

### 1. Preparation of Expression Vector for Protein Biosynthesis

According to the vector schematic view described in Table 1 below, chimeric AMO (AMO(amoB1)+sMMO(MMORF)), AMO-mimics(AMO-m1 to AMO-m2), BMO-mimics(BMO-m1 to BMO-m2) were fabricated through PCR. All the fabricated plasmid expression vectors were purified on an agarose gel, followed by investigating the sequence through complete DNA sequencing.

The PCR products prepared as described above were sequentially inserted into pT7-7 and pET28a expression vectors to construct expression vectors capable of expressing each protein.

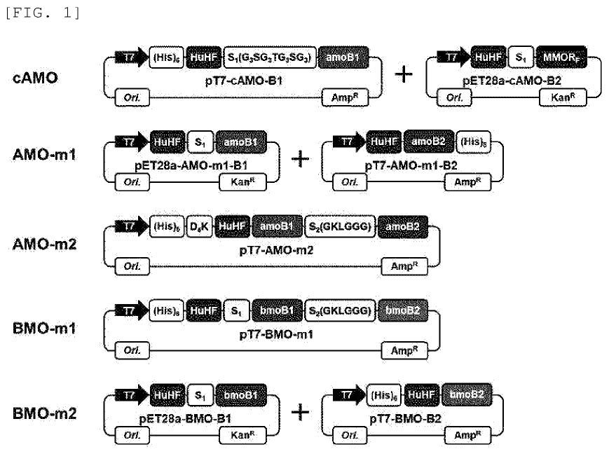

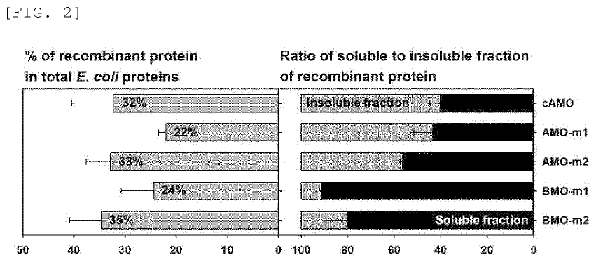

Vectors for expression of individual proteins were performed to pT7-cAMO-B1, pET28a-cAMO-B2, pET28a-AMO-m1-B1, pT7-AMO-m1-B2, pT7-AMO-m2, pT7-BMO-m1, pET28a-BMO-m2-B1 and pT7-BMO-m2-B2, respectively (FIG. 1).

TABLE 1

| Recombinant | |
|---|---|
| protein | Expression vector |
| cAMO | 1. NH₂-NdeI-H₆-huHF-S₁(G₃SG₃TG₃SG₃)-BamHI- |
| | amoB1(H38-L177)-HindIII-COOH |
| | 2. NH₂-NdeI-huHF-S₁-BamHI-MMOR_{F}(C99-A348)- |
| | HindIII-COOH |
| AMO-m1 | 1. NH₂-NdeI-huHF-S₁-BamHI-amoB1-HindIII- |
| | COOH2•NH₂-NdeI-huHF-BamHI-amoB2(Q270-L420)-H₆- |
| | HindIII-COOH |
| AMO-m2 | NH₂-NdeI-H₆-D₄K -huHF-BamHI-amoB1(H38-L177)- |
| | S₂(GKLGGG)- |
| | amoB2(Q270-L420)-HindIII-COOH |
| BMO-m1 | NH₂-NdeI-H₆-huHF-S₁-BamHI-bmoB1(H41-L180)- |
| | S₂(GKLGGG)- |
| | bmoB2(Q272-H418)-HindIII-COOH |
| BMO-m2 | 1. NH₂-NdeI-huHF-S₁-BamHI-bmoB1-HindIII- |
| | COOH2•NH₂-NdeI-H₆-huHF-BamHI-bmoB2-HindIII- |
| | COOH |

The sequences of the used proteins (domains) are shown in Table 2 below.

TABLE 2

| | | SEQ |
|---|---|---|
| | | ID |
| Domain | Sequence | NO. |
| amoB1 | HGERSQEPFLRMRTVQWYDIKWGPEVTKVNENAKITGKFHLAEDWPRAA | 1 |
| (38-177) | AQPDFSFFNVGSPSPVFVRLSTKINGHPWFISGPLQIGRDYEFEVNLRA | |
| | RIPGRHHMHAMLNVKDAGPIAGPGAWMNITGSWDDFTNPLKL | |
| amoB2 | QAGQSKVAALPVAPNPVSIVITDANYDVPGRALRVTMEVINNGDIPVTF | 2 |
| (270-420) | GEFTTAGIRFINSTGRKYLDPQYPRELIAVGLNFDDESAIQPGQTKELK | |
| | MEAKDALWEIQRLMALLGDPESRFGGLLMSWDAEGNRHINSIAGPVIPV | |
| | FTKL | |
| bmoB1 | HGEESQQAFQRTSTVVFYDVKFSDDTVDVGESVTITGMVRVMKSWPDHT | 3 |
| (41-180) | LEPPEMGYLTVSTPGPVFYVQEREMSGEFTPQSVRIEKGATYPFKLVIK | |
| | ARQPGTWHVHPGFGVEGAGTLVGAGKDITVNDTGVFENTVTL | |
| bmoB2 | QVVRTTPVPLAEEEVSGAVAPEIESIRFNAEADTLTMKLRVENTGAAAV | 4 |
| (272-418) | RLQRVQFGDVEFVSPSFASAADPDAQAMTVTPDQAIEPGGSATFTVEIQ | |
| | SEDLIVRSLVPVNEAELRVTGLLFFEDETGEQVVSEVNELTSAILQDFH | |
| MMOR_{F} | CRISFGEVGSFEAEVVGLNWVSSNTVQFLLQKRPDECGNRGVKFEPGQF | 5 |
| (99-348) | MDLTIPGTDVSRSYSPANLPNPEGRLEFLIRVLPEGRFSDYLRNDARVG | |
| | QVLSVKGPLGVFGLKERGMAPRYFVAGGTGLAPVVSMVRQMQEWTAPNE | |
| | TRIYFGVNTEPELFYIDELKSLERSMRNLTVKACVWHPSGDWEGEQGSP | |
| | IDALREDLESSDANPDIYLCGPPGMIDAACELVRSRGIPGEQVFFEKFL | |
| | PSGAA | |

### 2. Biosynthesis and Purification of Recombinant Proteins

E. coli strain BL21 (DE3) [F⁻ompThsdS_{B} (rB⁻mB−)], and pGro7/BL21 (DE3) [F⁻ompThsdS_{B} (rB⁻mB−)] were transformed with the expression vectors prepared above, respectively. For cAMO and BMO-m2 except for AMO-mimics and BMO-m1, E. coli strain BL21 was simultaneously transformed with two expression vectors, and transformants resistant to ampicillin and kanamycin were selected. The transformed E. coli was cultured in a flask (250 mL Erlenmeyer flask, 37° C., 150 rpm) including 50 mL of Luria-Bertani (LB) medium (containing 100 mg L⁻¹ ampicillin, 100 mg L⁻¹ kanamycin and 0.4 mM CuSO₄).

For AMO-m1, two expression vectors were simultaneously transformed into pGro7/BL21, and transformants resistant to ampicillin, kanamycin and chloroamphenicol were selected. The transformed E. coli was cultured in a flask (250 mL Erlenmeyer flask, 37° C., 150 rpm) including 50 mL of Luria-Bertani (LB) medium (containing 100 mg L⁻¹ ampicillin, 100 mg L⁻¹ kanamycin, 20 mg L⁻¹ chloroamphenicol, 0.5 g L⁻¹ arabinose and 0.4 mM CuSO₄).

For AMO-m2 and BMO-m1, an expression vector was transformed into BL21, and a transformant resistant to ampicillin was selected. The transformed E. coli was cultured in a flask (250 mL Erlenmeyer flask, 37° C., 150 rpm) including 50 mL of Luria-Bertani (LB) medium (containing 100 mg L⁻¹ ampicillin and 0.4 mM CuSO₄).

When the medium turbidity (OD₆₀₀) reached about 0.6, Isopropyl-β-D-thiogalactopyanosid (IPTG) (1 mM) was added to induce gene expression. After 14 hours of incubation at 20° C., the cultured E. coli was centrifuged at 5,000 rpm for 5 minutes to recover the cell precipitate, which in turn was suspended in 5 mL of a lysis solution (50 mM NaH₂PO₄, 300 mM NaCl, 10 mM imidazole, pH 8.0) and crushed using an ultrasonic crusher (Branson Ultrasonics Corp., Danbury, CT, USA). After crushing, centrifugation was performed at 13,000 rpm for 10 minutes to separate the supernatant and insoluble aggregates.

The separated supernatant was first subjected to Ni²⁺-NTA affinity chromatography using the binding of histidine and nickel fused to the recombinant protein, and then the recombinant protein was concentrated and buffer-exchanged to obtain purified recombinant protein. Details of each step are as follows.

### 1) Ni²⁺-NTA Affinity Chromatography

In order to purify the recombinant protein, E. coli cultured in the same manner as specified above was recovered, cell pellets thereof were resuspended in 5 mL of a lysis solution (50 mM NaH₂PO₄, 300 mM NaCl, 10 mM imidazole, pH 8.0), and an ultrasonic crusher was used to crush cells. The crushed cell solution was centrifuged at 13,000 rpm for 10 minutes to separate only the supernatant, and then each recombinant protein was separated using a Ni²⁺-NTA column (Quiagen, Hilden, Germany) (Wash Buffer: 50 mM NaH₂PO₄, 300 mM NaCl, 50 mM imidazole, pH 8.0/Elution Buffer: 50 mM NaH₂PO₄, 300 mM NaCl, 250 mM imidazole, pH 8.0)

### 2) Concentration and Buffer Exchange

2 mL of recombinant protein eluted through Ni²⁺-NTA affinity chromatography was placed in an ultracentrifugal filter (Amicon Ultra 100K, Millipore, Billerica, MA) and centrifuged at 5,000 rpm until 1 ml of solution remained on the column. Thereafter, the buffer was exchanged with Tris buffer (20 mM Tris-HCl, 250 mM NaCl, pH 8.0).

3. Analysis of Expression Rate and Cytoplasmic Solubility of Recombinant Proteins Including Prepared cAMO, AMO-, and BMO-Mimics

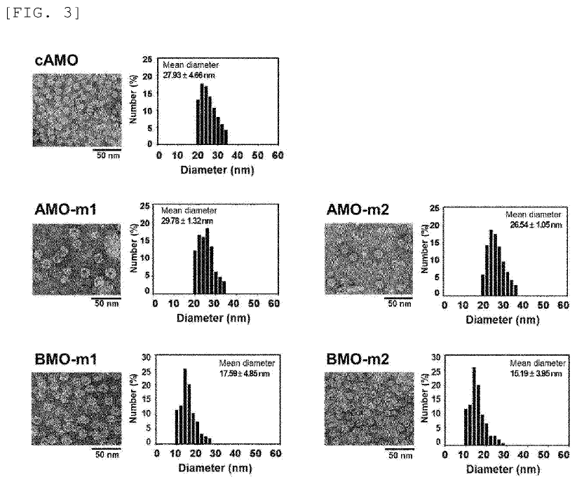

After the above process, the expression rate and cytoplasmic solubility of the purified recombinant protein were analyzed by SDS-PAGE. The supernatant (soluble fraction, sol), insoluble fraction (insol), and the purified recombinant protein obtained by centrifugation of the crushed cell solution of the recombinant protein were subjected to SDS-PAGE using 12% Tris-glycine precast gel (Invitrogen, California, U.S.A.). Then, the gel was stained with a Coomassie blue staining solution, and the expression rate and cytoplasmic solubility of each recombinant protein were analyzed using a densitometer (GS-800 Calibrated Densitometer, Bio-Rad, California, U.S.A.) for the stained protein band (FIG. 2).

4. Structural Analysis of Recombinant Proteins Including Prepared cAMO, AMO-, and BMO-Mimics

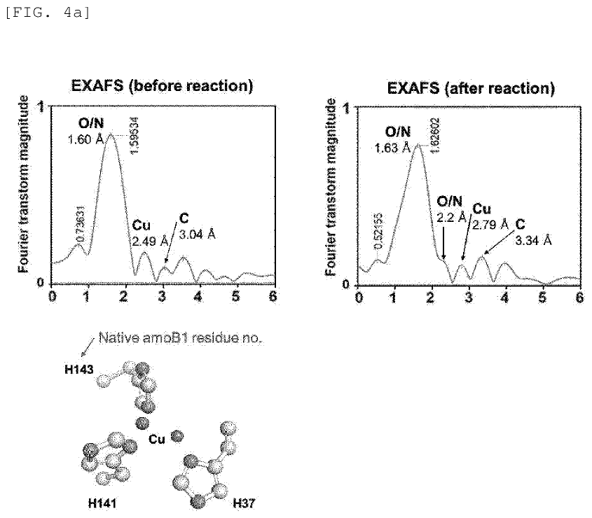

After the above process, transmission electron microscopy (TEM) imaging was performed to analyze the structure of the purified recombinant protein. To obtain stained images of proteins, electron microscope grids including naturally dried samples were incubated with 2% (w/v) aqueous uranyl acetate solution for 1 hour at room temperature. The protein image was observed using a Tecnai 20 (FEI, Hillsboro, Oreon, U.S.A.) electron microscope operating at 200 kV, and as a result of observation, it was confirmed that spherical nanoparticles were formed. Additionally, through dynamic light scattering (DLS) analysis, it was confirmed that spherical nanoparticles are formed with sizes of 27.9±4.7 nm for cAMO, 29.8±1.3 nm for AMO-m1, 26.5±1.1 nm for AMO-m2, 17.6±4.9 nm for BMO-m1, and 15.2±4.0 nm for BMO-m2, respectively (FIG. 3).

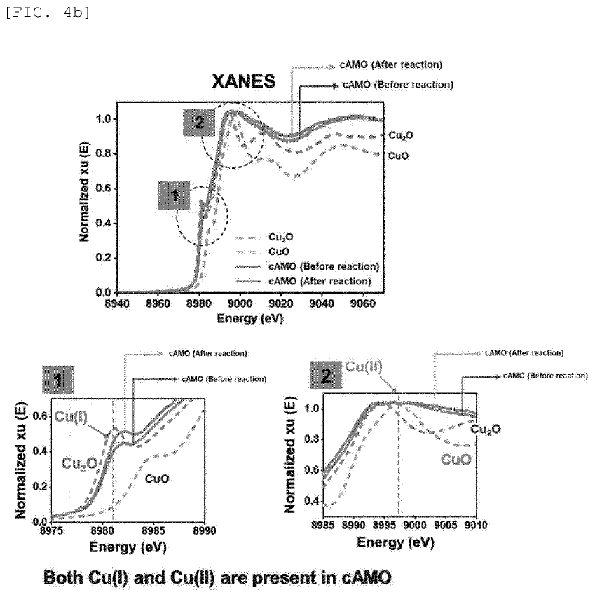

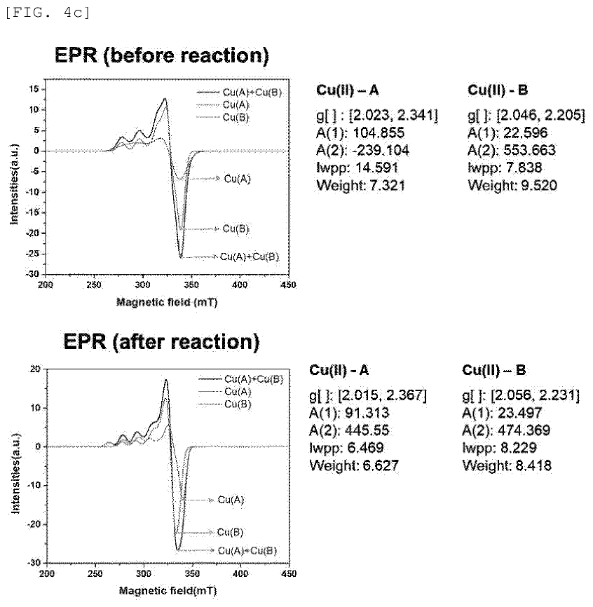

For structural analysis of the prepared cAMO recombinant protein, X-ray absorption spectroscopy (XAS) and electron paramagnetic resonance (EPR) spectra analysis were performed. For X-ray absorption near-edge structure (XANES), extended X-ray absorption fine structure (EXAFS) and EPR analysis of proteins, the solvent-exchanged samples with Tris buffer were pre-frozen at −80° C. for 3 hours, and the pre-frozen samples were lyophilized at −110° C. using a lyophilizer (FDU-2100, DRC-1000, EYELA). XAS analysis was performed for measurement by the XAFS beam line (BL11S2) of the Aichi Synchrontron Radiation Center (Aichi). A result of cAMO EXAFS analysis, information on a distance between copper ions and surrounding ligands present in the active site was confirmed. In the case of the sample subjected to the methane oxidation reaction, it was confirmed that the ligand distance was changed compared to the non-reacted sample, and one peak (up to 2.2 Å) was additionally observed. Further, as a result of XANES analysis, it was confirmed that mono- and bivalent copper ions (Cu(I), Cu(II)) were mixed. Additionally, through EPR analysis, it was confirmed that the bivalent copper ions existed in a valence-scrambled state (FIGS. 4a-c).

5. Verification of Methane and Butane Gas Oxidation Activity of Recombinant Proteins Including cAMO, AMO-, and BMO-Mimics

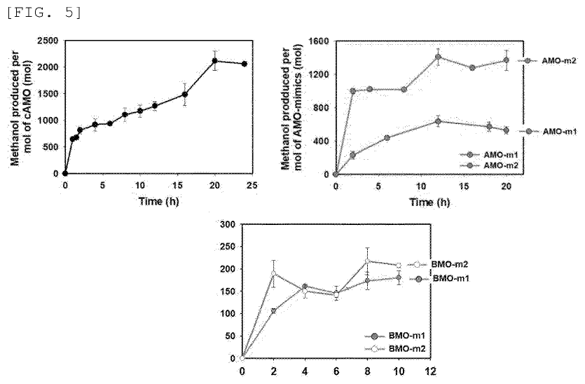

To verify the methane gas and butane gas oxidation activity of the purified recombinant protein, 1 mL of a recombinant protein solution containing a reducing agent NADH (0.2 mM) or duroquinol (0.35 mM) was injected to a 20 mL septa-sealed vial (catalogue no. 5182-0837, Agilent). For the methane and butane oxidation reaction through the recombinant protein, 19 mL of air in a headspace was removed through a syringe and 15 mL of methane or butane gas and 4 mL of air were injected. Then, the vial was immediately subjected to the enzymatic reaction in an incubator at 30° C. for maximum 24 hours. Further, an amount of methanol or butanol as an oxidation product generated by the enzymatic reaction was measured through gas chromatography (7890B GC, Agilent) to calculate the cumulative production (FIG. 5).

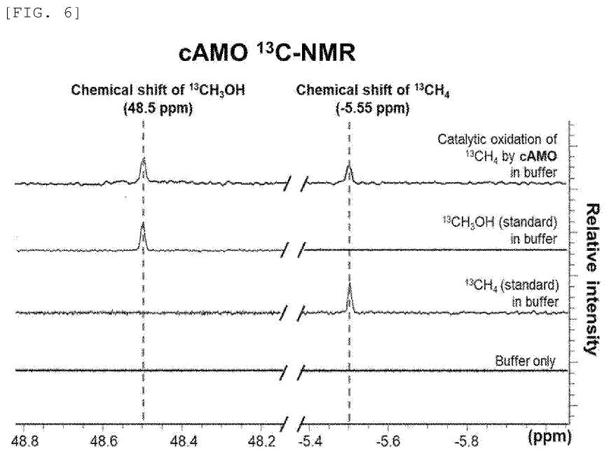

To verify the ¹³C-methane gas oxidation activity of cAMO, the enzymatic reaction was performed in the same manner as the methane oxidation reaction specified above, except that the methane gas was replaced with ¹³C-methane gas. For nuclear magnetic resonance (NMR) analysis, 5 vials after completing the reaction were heated at 80° C. for 15 minutes, followed by directly injecting 19 ml of headspace gas into 600 μl of sufficiently cooled ethanol using a syringe. Thereafter, 60 μl of ethanol-d₆ was added, transferred to an NMR tube (NORS55007, Sigma Aldrich), and ¹³C-methanol generated by the cAMO enzymatic reaction (which is the generated oxidation product) was confirmed by NMR analysis (FIG. 6).

1. A protein comprising self-assembled ferritin monomers, in which an ammonia oxidase active domain having methane oxidation activity, or a butane oxidase active domain having butane oxidation activity is fused.

2. The protein according to claim 1, wherein the ammonia oxidase active domain is selected from amoB1 (Ammonia monooxygenase beta subunit_domain 1) and amoB2 (Ammonia monooxygenase beta subunit_domain 2).

3. The protein according to claim 2, wherein the amoB1 consists of an amino acid sequence of SEQ ID NO: 1, and the amoB2 consists of an amino acid sequence of SEQ ID NO: 2.

4. The protein according to claim 2, wherein ferritin monomers, in which amoB1 and amoB2 are fused, are self-assembled.

5. The protein according to claim 2, wherein a ferritin monomer fused with amoB1 and a ferritin monomer fused with amoB2 are self-assembled.

6. The protein according to claim 1, wherein the butane oxidase active domain is selected from bmoB1 (Particulate Butane monooxygenase subunit B_domain 1) and bmoB2 (Particulate Butane monooxygenase subunit B_domain 2).

7. The protein according to claim 6, wherein the bmoB1 consists of an amino acid sequence of SEQ ID NO: 3, and the bmoB2 consists of an amino acid sequence of SEQ ID NO: 4.

8. The protein according to claim 6, wherein ferritin monomers, in which bmoB1 and bmoB2 are fused, are self-assembled.

9. The protein according to claim 6, wherein a ferritin monomer fused with bmoB1 and a ferritin monomer fused with bmoB2 are self-assembled.

10. The protein according to claim 1, wherein the ferritin monomer is a human ferritin heavy chain monomer.

11. The protein according to claim 10, wherein each domain is fused to any one selected from the group consisting of: inside α-helix of the ferritin monomer; between adjacent α-helices; N-terminus; C-terminus; A-B loop; B-C loop; C-D loop; D-E loop; between N-terminus and A helix; and between E helix and C-terminus.

12. A microorganism for expressing the protein according to claim 1.

13. The microorganism according to claim 12, wherein the microorganism is introduced with a vector which includes: a gene encoding a ferritin monomer; and a gene encoding an ammonia oxidase active domain selected from amoB1 (Ammonia monooxygenase beta subunit_domain 1) and amoB2 (Ammonia monooxygenase beta subunit_domain 2), or a butane oxidase active domain selected from bmoB1 (Particulate Butane monooxygenase subunit B_domain 1) and bmoB2 (Particulate Butane monooxygenase subunit B_domain 2).

14. The microorganism according to claim 12, wherein the microorganism is E. coli.

15. A composition for preparing methanol, comprising the protein according to claim 1, wherein the protein is fused with an ammonia oxidase active domain.

16. The composition according to claim 15, further comprising a reducing agent.

17. The composition according to claim 15, wherein the reducing agent is duroquinol.

18. A method for production of methanol, comprising reacting the composition according to claim 15 with methane gas.

19. A composition for preparing butanol, comprising the protein according to claim 1, wherein the protein is fused with a butaneoxidase active domain.

20. The composition according to claim 19, further comprising a reducing agent.

21. The composition according to claim 19, wherein the reducing agent is duroquinol.

22. A method for production of butanol, comprising reacting the composition according to claim 19 with butane gas.
